(19) United States
(12) Patent Application Publication
(10) Pub. No.: US 2024/0277512 A1
(43) Pub. Date: Aug. 22, 2024

(54) ABSORBENT BED PADDING DEVICE AND METHOD OF USE

(71) Applicant: Jeanette Hunter, Rosedale, NY (US)

(72) Inventor: Jeanette Hunter, Rosedale, NY (US)

(21) Appl. No.: 18/112,584

(22) Filed: Feb. 22, 2023

(51) Int. Cl. A61F 5/48

(52) U.S. Cl. CPC A61F 5/485

(57) ABSTRACT

An absorbent bed padding device for reducing incidence of urine induced skin damage and of pressure ulcers includes a pad, which is positionable atop a mattress. The pad comprises a lower layer and an upper layer, which is attached to and which substantially covers an upper face of the lower layer. The lower layer comprises an absorbent and resiliently compressible foam. The upper layer comprises a wicking and resiliently compressible foam, which wicks urine that is excreted by a user into the lower layer to reduce incidence of urine induced skin damage. The upper layer and the lower layer have a combined thickness that is sufficient to reduce pressure upon pressure points of the user's body. Thus, the pad reduces incidence of pressure ulcers.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM.

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to bed pads and more particularly pertains to a new bed pad for reducing incidence of urine induced skin damage and of pressure ulcers. The present invention discloses a bed pad that wicks urine away from a user's body while cushioning pressure points of the user's body.

### (2) Description of Related Art Including Information Disclosed Under 37 CFR 1.97 and 1.98

The prior art relates to bed pads, many of which are disclosed that comprise absorbent pads, mats, or the like. What is lacking in the prior art is a bed pad comprising upper and lower layers, both of which are resiliently compressible so as to cushion pressure points of the user's body, with the upper and lower layers being wicking and absorbent, respectively, so as to wick urine away from the user's body.

### BRIEF SUMMARY OF THE INVENTION

An embodiment of the disclosure meets the needs presented above by generally comprising a pad, which is configured to be positioned atop a mattress. The pad comprises a lower layer and an upper layer, which is attached to and which substantially covers an upper face of the lower layer. The lower layer comprises an absorbent and resiliently compressible foam. The upper layer comprises a wicking and resiliently compressible foam and thus is configured to wick urine that is excreted by a user into the lower layer to reduce incidence of urine induced skin damage. The upper layer and the lower layer have a combined thickness that is sufficient to reduce pressure upon pressure points of the user's body. Thus, the pad is configured to reduce incidence of pressure ulcers.

Another embodiment of the disclosure includes a method of reducing urine induced skin damage and pressure ulcers in a substantially bedridden user. Provision steps of the method providing bed that includes a mattress and providing an absorbent bed padding device, according to the disclosure above. A preparatory step of the method is positioning the pad atop the mattress. Use steps of the method are positioning a user upon the pad, allowing the pad to reduce pressure upon pressure points of the user's body, and allowing the upper layer to wick urine away from the user into the lower layer.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a side view of an absorbent bed padding device according to an embodiment of the disclosure.

FIG. 2 is an isometric perspective view of an embodiment of the disclosure.

FIG. 3 is an end view of an embodiment of the disclosure.

FIG. 4 is a top view of an embodiment of the disclosure.

FIG. 5 is a bottom view of an embodiment of the disclosure.

FIG. 6 is an in-use view of an embodiment of the disclosure.

FIG. 7 is a flow diagram for a method utilizing an embodiment of the disclosure.

### DETAILED DESCRIPTION OF THE INVENTION

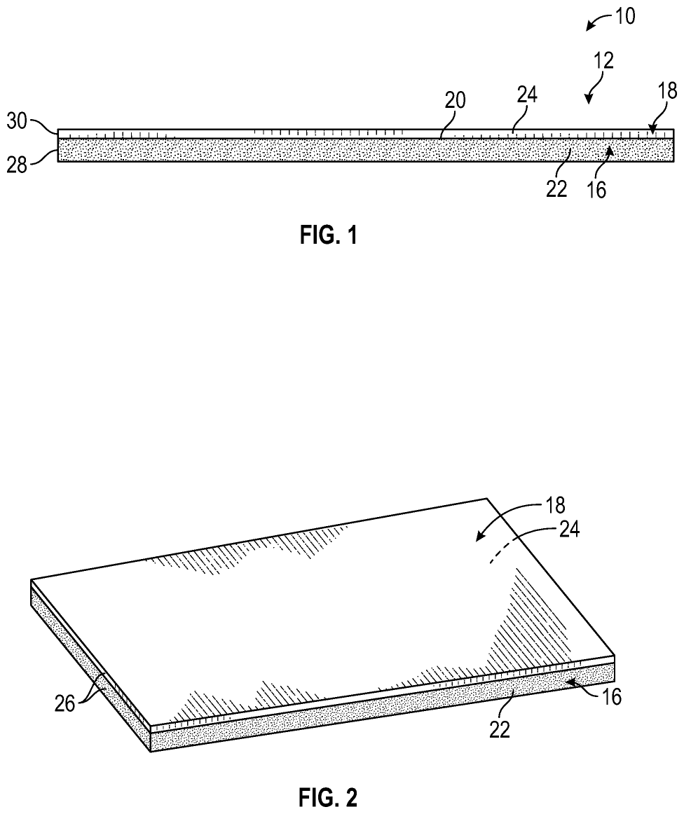

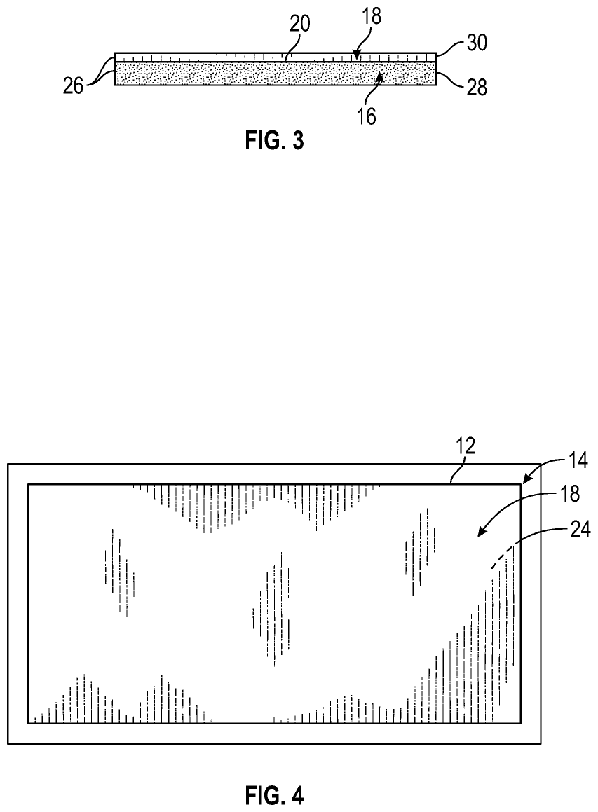

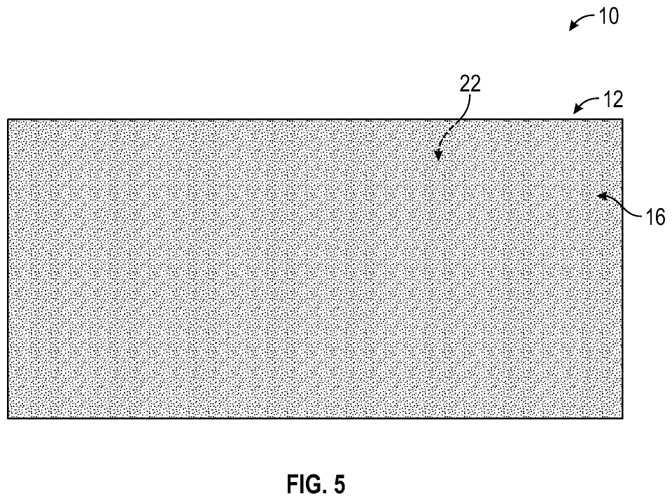

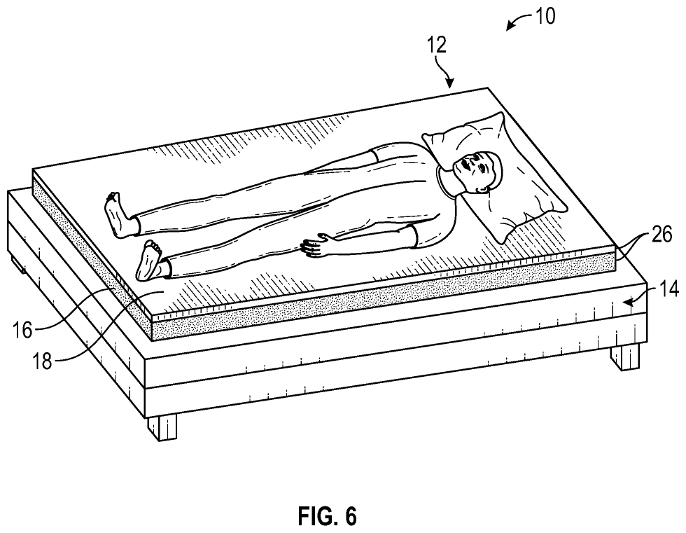

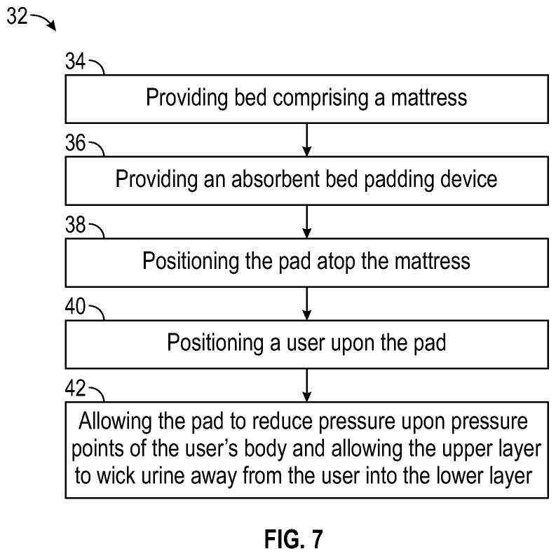

With reference now to the drawings, and in particular to FIGS. 1 through 7 thereof, a new bed pad embodying the principles and concepts of an embodiment of the disclosure and generally designated by the reference numeral 10 will be described.

As best illustrated in FIGS. 1 through 7, the absorbent bed padding device 10 generally comprises a pad 12, which is configured to be positioned atop a mattress 14. The pad 12 comprises a lower layer 16 and an upper layer 18, which is attached to and which substantially covers an upper face 20 of the lower layer 16. The lower layer 16 comprises an absorbent and resiliently compressible foam 22, such as open cell flexible polyurethane foam or other type of open cell absorbent foam. The upper layer 18 comprises a wicking and resiliently compressible foam 24, such as, but not limited to, one or more of polypropene, polyester, wool, or the like. The upper layer 18 thus is configured to wick urine that is excreted by a user into the lower layer 16 to reduce incidence of urine induced skin damage.

The upper layer 18 and the lower layer 16 have a combined thickness 26 that is sufficient to reduce pressure upon pressure points of the user's body. To achieve this reduction in pressure, the lower layer 16 has a thickness 28 of 2.54 to 12.70 cm and the upper layer 18 has a thickness 30 of 2.54 to 7.62 cm. The lower layer 16 may have a thickness 28 of 7.62 to 10.16 cm and the upper layer 18 may have a thickness 30 of 3.81 to 6.35 cm. Thus, the pad 12 is configured to reduce incidence of pressure ulcers.

The absorbent bed padding device 10 is anticipated to be useful for user's who are substantially bedridden, either temporarily or permanently, and who also suffer from incontinence. Pressure ulcers are a significant cause of morbidity for such user's and typically occur where bones are close to the surface, such as shoulders, elbows, midback, tail bones, hips, ankles, heals, and knees. Pressure ulcers are notoriously difficult to heal and can lead to infections, loss of limb, and death. Healing of pressure ulcers is hampered by repeated or prolonged exposure to urine, thus the absorbent bed padding device 10 provides benefit both in reducing incidence of pressure ulcers and in facilitating their healing.

As shown in FIG. 4, the lower layer 16 is substantially rectangularly shaped, although the present invention also anticipates the lower layer 16 being alternatively shaped, such as, but not limited to, square, oval, circular, or the like. Additionally, the present invention anticipates one or both of the lower layer 16 and the upper layer 18 being impregnated with a compound, such as citric acid, acetic acid, or the like, to neutralize the urea in the urine.

In use, the absorbent bed padding device enables a method of reducing urine induced skin damage and pressure ulcers in a substantially bedridden user 32. The method 32 comprises a first provision step 34, which entails providing a bed that includes a mattress 14. A second provision step 36 of the method 32 is providing an absorbent bed padding device 10, according to the specification above. A preparatory step 38 of the method 32 is positioning the pad 12 atop the mattress 14. A first use step 40 of the method 32 is positioning a user upon the pad 12. A second use step 42 of the method 32 is allowing the pad 12 to reduce pressure upon pressure points of the user's body and allowing the upper layer 18 to wick urine away from the user into the lower layer 16.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. An absorbent bed padding device comprising a pad configured for positioning atop a mattress, the pad comprising:
a lower layer comprising an absorbent and resiliently compressible foam; and
an upper layer attached to and substantially covering an upper face of the lower layer, the upper layer comprising a wicking and resiliently compressible foam, wherein the upper layer is configured for wicking urine excreted by a user into the lower layer for reducing incidence of urine induced skin damage, the upper layer and the lower layer have a combined thickness sufficient for reducing pressure upon pressure points of the user's body wherein the pad is configured for reducing incidence of pressure ulcers.

2. The absorbent bed padding device of claim 1, wherein the lower layer is substantially rectangularly shaped.

3. The absorbent bed padding device of claim 1, wherein:
the lower layer comprises open cell flexible polyurethane foam; and
the upper layer comprises one or more of polypropene, polyester, and wool.

4. The absorbent bed padding device of claim 1, wherein:
the lower layer has a thickness of 2.54 to 12.70 cm; and
the upper layer has a thickness of 2.54 to 7.62 cm.

5. The absorbent bed padding device of claim 4, wherein:
the lower layer has a thickness of 7.62 to 10.16 cm; and
the upper layer has a thickness of 3.81 to 6.35 cm.

6. An absorbent bed padding device comprising a pad configured for positioning atop a mattress, the pad comprising:
a lower layer comprising an absorbent and resiliently compressible foam, the lower layer comprising open cell flexible polyurethane foam, the lower layer having a thickness of 2.54 to 12.70 cm, the lower layer having a thickness of 7.62 to 10.16 cm, the lower layer being substantially rectangularly shaped; and
an upper layer attached to and substantially covering an upper face of the lower layer, the upper layer comprising a wicking and resiliently compressible foam, wherein the upper layer is configured for wicking urine excreted by a user into the lower layer for reducing incidence of urine induced skin damage, the upper layer and the lower layer have a combined thickness sufficient for reducing pressure upon pressure points of the user's body wherein the pad is configured for reducing incidence of pressure ulcers, the upper layer comprising one or more of polypropene, polyester, and wool, the upper layer having a thickness of 2.54 to 7.62 cm, the upper layer having a thickness of 3.81 to 6.35 cm.

7. A method of reducing urine induced skin damage and pressure ulcers in a substantially bedridden user comprising the steps of:
providing bed comprising a mattress;
providing an absorbent bed padding device comprising a pad, the pad comprising:
a lower layer comprising an absorbent and resiliently compressible foam, and
an upper layer attached to and substantially covering an upper face of the lower layer, the upper layer comprising a wicking and resiliently compressible foam, wherein the upper layer is configured for wicking urine excreted by a user into the lower layer for reducing incidence of urine induced skin damage, the upper layer and the lower layer have a combined thickness sufficient for reducing pressure upon pressure points of the user's body wherein the pad is configured for reducing incidence of pressure ulcers;
positioning the pad atop the mattress;
positioning a user upon the pad; and
allowing the pad to reduce pressure upon pressure points of the user's body and allowing the upper layer to wick urine away from the user into the lower layer.

8. The method of claim 7, wherein the lower layer is substantially rectangularly shaped.

9. The method of claim 7, wherein:
the lower layer comprises open cell flexible polyurethane foam; and
the upper layer comprises one or more of polypropene, polyester, and wool.

10. The method of claim 7, wherein:
the lower layer has a thickness of 2.54 to 12.70 cm; and
the upper layer has a thickness of 2.54 to 7.62 cm.

11. The method of claim 10, wherein:
the lower layer has a thickness of 7.62 to 10.16 cm; and
the upper layer has a thickness of 3.81 to 6.35 cm.
